SEQUENCE LISTING (19 sequences)

SEQ ID NO: 1 - DNA, 1785 bp, Brassica napus
agcgtaacggaccacaaaagaggatccatacaaatacatctcatcgcttcctctactattctccgacacacacactgagcaatgacgtccattaacgtaaagctcctttaccattacgtcataaccaaccttttcaacctttgcttctttccgttaacggcgatcgtcgccggaaaagcctatcggcttaccatagacgatcttcaccacttatactattcctatctccaacacaacctcataaccatcgctccactctttgccttcaccgttttcggttcggttctctacatcgcaacccggcccaaaccggtttacctcgttgagtactcatgctaccttccaccaacgcattgtagatcaagtatctccaaggtcatggatatcttttatcaagtaagaaaagctgatccttctcggaacggcacgtgcgatgactcgtcgtggcttgacttcttgaggaagattcaagaacgttcaggtctaggcgatgaaactcacgggcccgaggggctgcttcaggtccctccccggaagacttttgcggcggcgcgtgaagagacggagcaagttatcattggtgcgctagaaaatctattcaagaacaccaacgttaaccctaaagatataggtatacttgtggtgaactcaagcatgtttaatccaactccatcgctctccgcgatggtcgttaacactttcaagctccgaagcaacgtaagaagctttaaccttggtggcatgggttgtagtgccggcgttatagccattgatctagcaaaggacttgttgcatgtccataaaaatacgtatgctcttgtggtgagcacagagaacatcacttataacatttacgctggtgataataggtccatgatggtttcaaattgcttgttccgtgttggtggggccgctattttgctctccaacaagcctggagatcgtagacggtccaagtacgagctagttcacacggttcgaacgcataccggagctgacgacaagtcttttcgttgcgtgcaacaaggagacgatgagaacggcaaaatcggagtgagtttgtccaaggacataaccgatgttgctggtcgaacggttaagaaaaacatagcaacgttgggtccgttgattcttccgttaagcgagaaacttctttttttcgttaccttcatgggcaagaaacttttcaaagataaaatcaaacattactacgtcccggatttcaaacttgctattgaccatttttgtatacatgccggaggcagagccgtgattgatgtgctagagaagaacctagccctagcaccgatcgatgtagaggcatcaagatcaacgttacatagatttggaaacacttcatctagctcaatatggtatgagttggcatacatagaagcaaaaggaaggatgaagaaaggtaataaagtttggcagattgctttagggtcaggctttaagtgtaacagtgcagtttgggtggctctaaacaatgtcaaagcttcgacaaatagtccttgggaacactgcatcgacagatacccggtcaaaattgattctgattcaggtaagtcagagactcgtgtccaaaacggtcggtcctaataaatgatgtttgctctctttcgtttctttttatttgttataataatttgatggctacgatgtttctcttgtttgttatgaataaagaatgcaatggtgttctagtatttgattgttttacatgtatgtatctcttatttacatgaaatttttaaacgcctaggaaaaaaaaaaaaaaaaaaa

SEQ ID NO: 2 - DNA, 1468 bp, Brassica napus
aagctttacaacgatacacaaaacttataaccgtaatcaccattcattaacttaactactatcacatgcattcatgaattgaaacgagaaggatgtaaatagttgggaagttatctccacgttgaagagatcgttagcgagagctgaaagaccgagggaggagacgccgtcaacacggacagagtcgtcgaccctcacatgaagtaggaggaatctccgtgaggagccagagagacgtctttggtcttcggtttcgatccttgatctgacggagaagacgagagaagtgcgactggactccgtgaggaccaacagagtcgtcctcggtttcgatcgtcggtattggtggagaaggcggaggaatctccgtgacgagccagagagatgtcgtcggtcttcggtttcgatccttgatctgacggagaagacgagagaagtgcgacgagactccgtgaggaccaacagagttgtcctcggtttcgatcgtcggtttcggcggagaaggcggaggaatctccgtgaggagccagagagacgtcgttggtcttcggtttcgatccttgatctgatggagaagacgagacaagtgggacgagactcaacgacggagtcagagacgtcgtcggtcttcggtttcggccgagaaggcggagtcggtcttcggtttcggccgagaaggcggaggagacgtcttcgatttgggtctctcctcttgacgaagaaaacaaagaacacgagaaataatgagaaagagaacaaaagaaaaaaaaataaaaataaaaataaaatttggtcctcttatgtggtgacacgtggtttgaaacccaccaaataatcgatcacaaaaaacctaagttaaggatcggtaataacctttctaattaattttgatttaattaaatcactctttttatttataaaccccactaaattatgcgatattgattgtctaagtacaaaaattctctcgaattcaatacacatgtttcatatatttagccctgttcatttaatattactagcgcatttttaatttaaaattttgtaaacttttttggtcaaagaacatttttttaattagagacagaaatctagactctttatttggaataatagtaataaagatatattaggcaatgagtttatgatgttatgtttatatagtttatttcattttaaattgaaaagcattatttttatcgaaatgaatctagtatacaatcaatatttatgttttttcatcagatactttcctattttttggcacctttcatcggactactgatttatttcaatgtgtatgcatgcatgagcatgagtatacacatgtcttttaaaatgcatgtaaagcgtaacggaccacaaaagaggatccatacaaatacatctcatcgcttcctctactattctccgacacacacactgagcaatgacgtccattaacgtaaagctcctttaccattacgtc

SEQ ID NO: 3 - protein, 506 aa, Brassica napus
MTSINVKLLYHYVITNLFNLCFFPLTAIVAGKAYRLTIDDLHHLYYSYLQHNLITIAPLFAFTVFGSVLYIATRPKPVYLVEYSCYLPPTHCRSSISKVMDIFYQVRKADPSRNGTCDDSSWLDFLRKIQERSGLGDETHGPEGLLQVPPRKTFAAAREETEQVIIGALENLFKNTNVNPKDIGILVVNSSMFNPTPSLSAMVVNTFKLRSNVRSFNLGGMGCSAGVIAIDLAKDLLHVHKNTYALVVSTENITYNIYAGDNRSMMVSNCLFRVGGAAILLSNKPGDRRRSKYELVHTVRTHTGADDKSFRCVQQGDDENGKIGVSLSKDITDVAGRTVKKNIATLGPLILPLSEKLLFFVTFMGKKLFKDKIKHYYVPDFKLAIDHFCIHAGGRAVIDVLEKNLALAPIDVEASRSTLHRFGNTSSSSIWYELAYIEAKGRMKKGNKVWQIALGSGFKCNSAVWVALNNVKASTNSPWEHCIDRYPVKIDSDSGKSETRVQNGRS

SEQ ID NO: 4 - protein, 506 aa, Brassica napus cv. Askari
MTSVNVKLLYHYVITNLFNLCFFPLTAIVAGKAYRLTIDDLHHLYYSYLQHNLITIAPLFAFTVFGSVLYIATRPKPVYLVEYSCYLPPTHCRSSISKVMDIFYQVRKADPSRNGTCDDSSWLDFLRKIQERSGLGDETHGPEGLLQVPPRKTFAAAREETEQVIIGALENLFKNTNVNPKDIGILVVNSSMFNPTPSLSAMVVNTFKLRSNVRSFNLGGMGCSAGVIAIDLAKDLLHVHKNTYALVVSTENITYNIYAGDNRSMMVSNCLFRVGGAAILLSNKPGDRRRSKYELVHTVRTHTGADDKSFRCVQQGDDENGKIGVSLSKDITDVAGRTVKKNIATLGPLILPLSEKLLFFVTFMGKKLFKDKIKHYYVPDFKLAIDHFCIHAGGRAVIDVLEKNLALAPIDVEASRSTLHRFGNTSSSSIWYELAYIEAKGRMKKGNKVWQIALGSGFKCNSAVWVALNNVKASTNSPWEHCIDRYPVKIDSDSGKSETRVQNGRS

SEQ ID NO: 5 - DNA, 21 bp, Artificial Sequence
  A primer
atgacgtccgttaacgttaag

SEQ ID NO: 6 - DNA, 20 bp, Artificial Sequence
  A primer
atcagctccagtatgcgttc

SEQ ID NO: 7 - DNA, 22 bp, Artificial Sequence
  A primer
cgttaacgtaaagctcctttac

SEQ ID NO: 8 - DNA, 22 bp, Artificial Sequence
  A primer
tagacctgaacgttcttgaatc

SEQ ID NO: 9 - DNA, 20 bp, Artificial Sequence
  A primer
tgacgtaatggtaaaggagc

SEQ ID NO: 10 - DNA, 18 bp, Artificial Sequence
  A primer
ttcaagctccgaagcaac

SEQ ID NO: 11 - DNA, 12 bp, Artificial Sequence
  5′ end of three independent clones
agcaatgacgtc

SEQ ID NO: 12 - DNA, 22 bp, Artificial Sequence
  A primer
aggatccatacaaatacatctc

SEQ ID NO: 13 - DNA, 21 bp, Artificial Sequence
  A primer
agagaaacatcgtagccatca

SEQ ID NO: 14 - DNA, 36 bp, Artificial Sequence
  A primer
ggaattcaaacaaatgacgtccgttaacgtaaagct

SEQ ID NO: 15 - DNA, 20 bp, Artificial Sequence
  A primer
tctagcgcaccaatgataac

SEQ ID NO: 16 - DNA, 23 bp, Artificial Sequence
  A primer
cggggtaccggcggccgctctag

SEQ ID NO: 17 - DNA, 31 bp, Artificial Sequence
  A primer
cgtggccagccggccatggtaattgtaaatg

SEQ ID NO: 18 - DNA, 21 bp, Artificial Sequence
  A primer
ctctcgaattcaatacacatg

SEQ ID NO: 19 - DNA, 27 bp, Artificial Sequence
  A primer
tcccccgggtgctcagtgtgtgtgtcg

(19) United States
(12) Patent Application Publication
(10) Pub. No.: US 2003/0159173 A1
(43) Pub. Date: Aug. 21, 2003

(54) Elongase promoters for tissue-specific expression of transgenes in plants

(76) Inventors: Frank P. Wolter, Alfter-Oedekoven (DE); Jixiang Han, Oxford, OH (US); Margrit Frentzen, Aachen (DE)

Correspondence Address: SCHWEGMAN, LUNDBERG, WOESSNER & KLUTH, P.A., P.O. BOX 2938, MINNEAPOLIS, MN, 55402, US

(21) Appl. No.: 10/126,447

(22) Filed: Apr. 19, 2002

Related U.S. Application Data
(63) Continuation of application No. PCT/EP00/10363, filed on Oct. 20, 2000.

(30) Foreign Application Priority Data
Oct. 20, 1999 (DE) 199 50 589.6

(51) Int. Cl. A01H 5/00; C12N 15/82; C12N 5/04; C07H 21/04; C12N 9/10

(52) U.S. Cl. 800/281; 536/23.2; 435/69.1; 435/419; 435/193; 800/306

(57) ABSTRACT

The invention relates to chimerical genes that have (i) a DNA sequence coding for a desired product, and (ii) an elongase promoter. The DNA sequence is functionally linked with the promoter to allow expression of the product under the control of the promoter. The invention further relates to vectors, plant cells, plants and plant parts and microorganisms that contain the chimerical gene and to methods for producing such vectors, plant cells, plants and plant parts and microorganisms. The invention also relates to elongase-encoding sequences from Brassica napus and to transgenic plants and microorganisms expressing said sequences.

The present invention relates to chimeric genes having (i) a DNA sequence encoding a desired product, and (ii) an elongase promoter, the DNA sequence being operatively linked with the promoter to allow expression of the product under the control of the promoter. The invention further relates to vectors, plant cells, plants and plant parts containing the chimeric gene, and to methods for producing such plant cells, plants and plant parts. The invention also relates to sequences from Brassica napus encoding active elongase enzymes, and to transgenic microorganisms and plants containing elongase-coding sequences. Furthermore, the invention relates to methods for shifting the chain length of fatty acids towards longer chain fatty acids in transgenic plants, and for producing longer chain polyunsaturated fatty acids in microorganisms and plants.

Long chain fatty acids comprising more than 18 carbon atoms and being denoted as very long chain fatty acids (VLCFAs) are very common in nature. These fatty acids are found mainly in seed oils of various plant species, where they are mostly found incorporated into triacylglycerides. VLCFAs in this form are found especially in Brassicaceae, Tropaeolaceae and Limnanthaceae. The seed oils of the Brassicaceae family, such as Brassica napus, Crambe abyssinica, Sinapsis alba, Lunaria annua, usually contain 40-60% erucic acid (cis-13-docosenic acid, 22:1_{Δ13}), whereas the Tropaeolaceae family may contain up to 80% erucic acid in the seed oil. The seed oils of the Limnanthes species or jojoba even contain more than 90% VLCFAs.

In seed oils, VLCFAs usually accumulate as monounsaturated cis-n-9 fatty acids such as 20:1^{Δ}, 22:1^{Δ13}, and 24:1^{Δ15}. However, some species may also contain VLCFAs of the cis-n-7 type such as 20:1^{Δ13} in Sinapsis alba and 20:1_{Δ5} which is predominant in the oil of Limnanthes species.

Application areas of vegetable fats and oils range from detergents and cleaning agents through cosmetics to dye additives, lubricating agents and hydraulic oils. In particular, a high content of erucic acid is regarded a breeding goal in classic as well as in modern plant breeding, since it is not only used as an anti-foaming agent in detergents or as an anti-blocking agent in the production of plastics, but erucic acid and its derivatives such as arachinic acid, pelagonic acid, brassylic acid and erucic acid amides, are used as preservation agents, flavouring agents, plastic softeners, formulation agents, flotation agents, wetting agents, emulsifiers, and lubricating agents as well.

VLCFAS are generated by successive transfer of C₂-units of malonyl-CoA to long chain acyl groups derived from de novo-synthesis of fatty acids in the plastids. These elongation reactions are catalysed by fatty acid elongases (FAE), each elongation cycle consisting of four enzymatic steps: (1) condensation of malonyl-CoA and a long chain acyl residue, resulting in generation of β-ketoacyl-CoA, (2) reduction of β-ketoacyl-CoA to β-hydroxyacyl-CoA, (3) dehydration of β-hydroxyacyl-CoA to trans-2,3-enoyl-CoA, (4) reduction of trans-2,3-enoyl-CoA, resulting in an elongated acyl-CoA. The condensation reaction, catalysed by a β-ketoacyl-CoA synthase (KCS), is the rate-determining step of the chain elongation.

VLCFAs are mainly enriched in seed triacylglycerides of most of the Brassica species such as Brassica napus. In developing oil seeds, triacylglycerides are synthesised by means of the Kennedy pathway, in which mainly the following four enzymatic reactions participate. First, glycerol-3-phosphate is acylated by acyl-CoA at position sn-1 to form lysophosphatidate (sn-1-acylglycerol-3-phosphate). This reaction is catalysed by an sn-glycerol-3-phosphate-acyltransferase (GPAT). Then, a second acylation step follows, catalysed by an sn-1-acylglycerol-3-phosphate-acyltransferase (lysophosphatidic acid acyltransferase, LPAAT) forming phosphatidate, which in the next step is transformed to diacylglycerol (DAG) by a phosphatidate phosphatase. Finally, DAG is acylated to a triacylglyceride at its sn-3 position by an sn-1,2-diacylglycerol-acyltransferase (DAGAT).

During the last years, KCS-genes were cloned from A. thaliana and jojoba. Transposon-tagging with the maize transposon activator allowed cloning of the fatty acid elongase gene 1 (FAE1), the product of which participates in the synthesis of VLCFAs (James et al. (1995) Plant Cell 7: 309-319). Furthermore, Lassner et al. managed to isolate a jojoba DNA clone from a developing seeds cDNA library (1996, Plant Cell 8: 281-292). Recently, the A. thaliana KCS-1 gene was cloned (Todd et al. (1999) Plant J. 17: 119-130). The isolation of a cDNA encoding a 3-ketoacyl-CoA synthase from Brassica napus was described 1997 by Clemens and Kunst (Plant Physiol. 115, 313-314); however, the cDNA sequence disclosed in the prior art does not seem to encode an active enzyme.

A β-ketoacyl-CoA synthase gene which encodes an active enzyme, or the tranfer of which to transgenic organisms in fact results in a detectable KCS activity, could so far not be successfully isolated from rapeseed, although rapeseed is the most important production facility of vegetable oils, and modem plant breeding therefore and for other reasons has a particularly strong interest in useful genes from just this crop.

Rapeseed has naturally high concentrations of erucic acid (˜50%), and rapeseed varieties with high contents of erucic acid (high erucic acid rapeseed, HEAR) are the main source of erucic acid as industrial food stock. However, in view of the high costs of erucic acid purification, the presently obtained content of 55% erucic acid in the seed oils from HEAR varieties is not sufficient to compete with alternative sources from petrochemicals. Increasing the erucic acid content in rapeseed oil by gene technological methods may solve this problem, and may markedly improve the industrial usefulness of rapeseed as an erucic acid producer. On the other hand, erucic acid is unwanted as a food component due to its unpleasant flavour and other negative characteristics, which in recent years has led to the breeding of rapeseed varieties with low erucic acid content (low erucic acid rapeseed, LEAR) which hardly contain any erucic acid in their seed oil at all. Rapeseed varieties can therefore be classified into industrially interesting HEAR-varieties and nutritionally advantageous LEAR-varieties.

One object of the present invention is to provide a β-ketoacyl-CoA-synthase gene or a corresponding method, by which the content of 22:1 fatty acids in plants and especially in oil seed can be increased particularly advantageously.

This object is solved by successful isolation and cloning of a KCS-gene from Brassica napus.

It was now unexpectedly found that KCS genes and especially the KCS gene from rapeseed described in the examples, are well suited for increasing the content of VLCFA and especially of 22:1 fatty acids in transgenic organisms, especially in oil seed plants. Here, not only the particularly high erucic acid content, which can be achieved by expression of the KCS gene in accordance with the invention, is advantageous compared to the prior art, but also the observed increase of the ratio of 22:1 fatty acids to the less desired 20:1 fatty acids.

Long chain fatty acids are of great relevance in the food sector and in the pharmaceutical sector. However, it is mainly the long chain polyunsaturated fatty acids (LC-PUFA), the essential relevance of which for the human health has recently become more and more obvious. They are fatty acids with two, but mainly three and more double bonds and chain lengths of 18 and more carbon atoms, but mainly chain lengths of 22 and 24. Important representatives are arachidonic acid (5,8,11,14-eicosatetraenoic acid), eicosapentaenoic acid (5,8,11,14,17-eicosapentaenoic acid, EPA) and docosapentaenoic acid (clupanodonic acid, 4,8,12,15,19-docosapentaenoic acid, DHA). Fish are the primary natural source of LC-PUFA. Considering the recently recognised high demand and the already dangerous overfishing of the oceans, the global demand may not be satisfied from this source on a continuous basis. Therefore, biotechnological production methods come to the fore. For this production, mainly microorganisms and plants may come into consideration. As microorganisms, yeast, fungi and bacteria may be particularly useful.

Biosynthesis of fatty acids starts with the common fatty acids linoleic acid and alpha-linolenic acid, and comprises alternating desaturation and elongation steps. Especially the desaturases required for the desaturation steps are being studied intensely, the genes of which were isolated mainly from marine microorganisms and are known to one skilled in the art. The required elongation steps represent a problem that has not been solved satisfyingly yet, since the elongase systems in the target organisms do not elongate these fatty acids at all or only insufficiently.

One further object of the present invention is therefore to provide a β-ketoacyl-CoA synthase gene and a corresponding method, by which PUFA may be elongated in microorganisms and in plants to the desired very long chain LC-PUFA species with 20 and more carbon atoms. In particular, the LC-PUFA are 18:2^{9,12}, 18:3^{9,12,15}, 18:3^{6,9,12,} 20:3^{8,11,14}, and 20:4^{5,8,11,14}.

The problem of the elongation of PUFA and particularly of very long chain PUFA by molecularbiological techniques and suitable genes has not been satisfyingly solved to date in the prior art.

This object is now solved by providing a method for production of longer chain polyunsaturated fatty acids by elongation of shorter chain polyunsaturated fatty acids in transgenic microorganisms and plants by elongation of polyunsaturated fatty acids, the elongation being catalysed by a β-ketoacyl-CoA synthase in the transgenic microorganisms or plants. Preferably, the KCS is an enzyme which is naturally present in rapeseed.

Thereby not only natural polyunsaturated fatty acids can be elongated, but also polyunsaturated fatty acids which are taken up from the environment by the microorganism or the plant. Furthermore, also polyunsaturated fatty acids generated in the target organism by gene technological modifications of the target organism, i.e. the microorganism or the plant, can be elongated by the enzymatic activity of a β-ketoacyl-CoA synthase. Very useful in this context is the co-expression of desaturase genes in the target organism, providing the desired polyunsaturated fatty acids as a substrate for the β-ketoacyl-CoA synthase. Of course, desaturase genes can also be co-expressed in the target organism together with other elongase genes in order to provide the desired polyunsaturated fatty acids with the desired chain length in the target organism.

Therefore, the invention relates to a method for producing longer chain polyunsaturated fatty acids (LC-PUFA) by elongation of shorter chain, polyunsaturated fatty acids in microorganisms, preferably bacteria, yeasts and fungi, and in plant cells by (i) elongation of naturally present polyunsaturated fatty acids or (ii) elongation of polyunsaturated fatty acids taken up from the environment, comprising the steps:

a) Generating a nucleic acid sequence in which a promoter region being active in the microorganism or in the plant cell is operatively linked with a nucleic acid sequence encoding a protein with β-ketoacyl-CoA synthase activity,

b) Transfer of the nucleic acid sequence from step (a) to microorganisms or plant cells,

c) In the case of plant cells, optionally regeneration of fully transformed plants, and

d) If desired, propagation of the generated transgenic organisms.

In the case of transgenic plant cells, it is not necessary to always generate fully transgenic plants. It may be desirable to perform the production of the long chain polyunsaturated fatty acids (LC-PUFA) in plant cells, such as in form of suspension cultures or callus cultures.

The observation is very surprising, that the KCS genes used in accordance with the invention and particularly the KCS gene from rapeseed, generate a gene product in transgenic organisms and cells which is able to elongate PUFA and particularly LC-PUFA. To date it has only been known that KCS plays a role in the elongation of saturated and monounsaturated fatty acids.

The nucleic acid encoding a protein with the activity of a β-ketoacyl-CoA synthase preferably is a nucleic acid sequence from Brassica napus. More preferably, it is a nucleic acid sequence comprising the sequence denoted in SEQ ID No. 1, or parts thereof. A person skilled in the art may learn other KCS genes from the literature and gene data bases. Thereby, the cDNA clone disclosed by Clemens and Kunst 1997 in Plant Physiol. (Vol. 115, page 113-114) with reference to accession no. AF009563, is explicitly excluded since the therein described cDNA sequence does not encode a protein with the activity of a KCS. The authors did not present evidence for KCS enzymatic activity; in fact, the prior art is restricted to the disclosure of the sequence accessible in accession no. AF009563.

In a special embodiment, such polyunsaturated fatty acids, in particular LC-PUFA, are elongated within the scope of the method in accordance with the invention, which are generated by gene technological manipulation in the target organism, wherein the gene technological manipulation may comprise the expression of desaturase genes and the expression of further elongase genes.

For the production of very long chain polyunsaturated fatty acids, such as arachidonic acid and eicosapentaenoic acid, Δ6- and Δ5-desaturase genes are required. Suitable genes were cloned from various organisms, and are available to those skilled in the art, see for example Sperling et al. (2000), Eur. J. Biochem. 267, 3801-3811; Cho et al. (1999). J. Biol. Chem. 274, 471-477; Sakoradani et al. (1999), Gene 238, 445-453; Sayanova et al. (1999), Journal of Experimental Botany 50, 1647-1652; Girke et al. (1998), The Plant Journal 15, 39-48; Huang et al. (1999), Lipids 34, 649-659; Saito et al. (2000), Eur. J. Biochem. 267, 1813-1818; Cho et al. (1999), J. Biol. Chem. 274, 37335-37339; Knutzon et al. (1998), J. Biol. Chem. 273, 29360-29366; Michaelson et al. (1998), J. Biol. Chem. 273, 19055-19059; Broun et al. (1999), Annu. Rev. Nutr. 19, 197-216; Napier et al. (1998), Biochem. J. 230, 611-614; Nunberg et al., (1996), Plant Physiol. 111 (Supplement), 132; Reddy et al. (1996), Nat. Biotechnol. 14, 639-642; Sayanova et al. (1997), Proc. Natl. Acad. Sci. USA 94, 4211-4216.

Depending on which long chain polyunsaturated fatty acid is desired, further genes, such as elongase genes, have to be transferred together with suitable desaturase genes. For example, for the production of docosapentaenoic acid (22:6), an elongase that catalyses the elongation from 22:5 into 24:5 should be expressed, together with a Δ6-desaturase providing the Δ6-desaturation to 24:6.

A person skilled in the art may easily learn suitable desaturase and elongase genes from the literature and gene data bases. Suitable genes for β-ketoacyl-CoA synthases being able to elongate γ-linoleic acid (GLA) have already been cloned from C. elegans and Mortierella alpina (see for example Das et al. (2000) 14^{th} International Symposium on Plant Lipids, Cardiff, Jul. 23/28, 2000 (“Polyunsaturated fatty acid-specific elongation enzymes”), Beaudoin et al. (2000), 14^{th} International Symposium on Plant Lipids, Cardiff, Jul. 23/28, 2000 (“Production of C20 polyunsaturated fatty acids by pathway engineering: Identification of a PUFA elongase component”); Beaudoin et al. (2000), Proc. Natl. Acad. Sci. USA 97, 5421-5426).

The invention therefore also relates to a method of producing longer chain polyunsaturated fatty acids (LC-PUFA) by elongation of shorter chain polyunsaturated fatty acids in microorganisms, preferably bacteria, yeasts and fungi, and in plant cells by elongation of polyunsaturated fatty acids, which are generated in the microorganism and in the plant cell, respectively, due to the expression of one or more introduced desaturase or/and elongase genes, comprising the steps:

a) Generating a nucleic acid sequence in which a promoter region being active in the microorganism or in the plant cell is operatively linked with a nucleic acid sequence encoding a protein with β-ketoacyl-CoA synthase activity,

b) transfer of the nucleic acid sequence from step a) to microorganisms or plant cells,

c) in the case of plant cells, optionally regeneration of fully transformed plants, and

d) if desired, propagation of the generated transgenic organisms.

The invention further relates to a method for altering the β-ketoacyl-CoA synthase activity in transgenic plants by transfer and expression of a nucleic acid sequence encoding a protein with β-ketoacyl-CoA synthase activity from Brassica napus. Preferably, the nucleic acid sequence encoding a protein with β-ketoacyl-CoA synthase activity comprises the sequence denoted in SEQ ID No. 1 or parts thereof.

Apart from bacteria, fungi and yeast, algae may also be used for application of the methods in accordance with the invention.

Furthermore, one object of the invention is to provide a new seed-specific promoter for the generation of transgenic plants with altered gene expression.

This object is solved by isolation and characterisation of a KCS promoter suitable for seed-specific expression of any coding region in plants. As demonstrated below, the KCS promoter is a particularly strong promoter, being particularly useful for tissue-specific expression of interesting genes in plants. The KCS promoter may be present in translational or transcriptional fusion with the desired coding regions and be transferred to plant cells. A person skilled in the art is able to perform both, the generation of suitable chimeric gene constructs and the transformation of plants with these constructs using standard methods. See for example Sambrook et al. (1998) Molecular Cloning: A Laboratory Manual, 2. Edition, Cold Spring Harbor Laboratory Press, Cold Spring Harbor, N.Y., or Willmitzer L. (1993) Transgenic Plants, in: Biotechnology, A Multi-Volume Comprehensive Treatise (H. J. Rehm, G. Reed, A. Pühler, P. Stadler, eds., Vol. 2, 627-659, V. C. H. Weinheim—New York—Basel—Cambridge. For generation of plants in accordance with the invention, several methods may be suitable. On the one hand, plants or plant cells may be modified by conventional gene technological transformation methods in such way that the new nucleic acid molecules can be integrated into the plant genome, e.g. stable transformants are generated. On the other hand, a nucleic acid molecule in accordance with the invention, the presence and optionally the expression of which in the plant cell cause a change in fatty acid content, may be present in the plant cell or in the plant as a self-replicating system. To prepare the introduction of foreign genes into higher plants, a number of cloning vectors are available, containing E. coli replication signals and a marker gene for selection of transformed bacterial cells. Examples of such vectors are pBR322, pUC series, M13mp series, pACYC184, etc. The desired sequence may be introduced into the vector through a suitable restriction site. The resulting plasmid may be used for transformation of E. coli cells. Transformed E. coli cells are cultivated in a suitable growth medium and subsequently harvested and lysed, and the plasmid is recovered. Generally, for characterisation of the recovered plasmid DNA, restriction site analysis, gel electrophoresis, and other biochemical and molecular biological methods may be employed as a method of analysis. After each manipulation, the plasmid DNA may be digested, and the recovered DNA fragments may be linked with other DNA sequences. For the introduction of DNA into a plant host cell, a number of suitable known techniques are available, whereby a person skilled in the art may be able to identify the individually most suitable method without difficulties. These techniques comprise the transformation of plant cells with T-DNA using Agrobacterium tumefaciens oder Agrobacterium rhizogenes as transformation means, protoplast fusion, direct gene transfer of isolated DNA in protoplasts, DNA electroporation, biolistic introduction of DNA, and other possibilities. For DNA injection and electroporation into plant cells, per se no special requirements exist regarding the used plasmids. This is true in a similar way for direct gene transfer. Simple plasmids, such as pUC derivatives, may be used. If whole plants are to be regenerated from such transformed cells, the presence of a selectable marker gene is required.

The person skilled in the art is familiar with gene selection markers, and will not have difficulties in selecting a suitable marker. Depending on the introduction method for desired genes into the plant cell, other DNA sequences may be required. If, for example, the Ti or Ri plasmid is used for the transformation of the plant cell, at least the right border, however more often both, the right and the left border of the T-DNA in the Ti or in the Ri plasmid, has to be linked as flanking region with the genes to be introduced. If agrobacteria are used for transformation, the DNA to be introduced has to be cloned into special plasmids, either into an intermediate or into a binary vector. Intermediate vectors may be integrated into the Ti or Ri plasmid of the agrobacteria by homologous recombination due to sequences which are homologous to sequences in the T-DNA. This also contains the vir region which is required for T-DNA transfer. Intermediate vectors are not able to replicate in agrobacteria. Supported by a helper plasmid, the intermediate vector may be transferred (conjugation) to Agrobacterium tumefaciens. Binary vectors are able to replicate in E. coli as well as in agrobacteria. They contain a selection marker gene, and a linker or polylinker framed by the right and left T-DNA border region. They may be transformed directly into agrobacteria. The agrobacterial host cell should contain a plasmid with a vir region. The vir region is required for the transfer of the T-DNA into the plant cell. Additional T-DNA may be present. The so transformed agrobacterium will be used for transformation of plant cells. The use of T-DNA for transformation of plant cells has been studied intensely, and is described sufficiently well in generally known reviews and plant transformation manuals. For transfer of the DNA into the plant cell, plant explantates may be cultivated together with Agrobacterium tumefaciens or Agrobacterium rhizogenes. From the infected plant material (e.g. leaf pieces, stem segments, roots, but also protoplasts or suspension-cultivated plant cells) whole plants may be regenerated in a suitable medium which may contain antibiotics or biocides for selection of transformed cells. Plant regeneration may take place according to conventional regeneration methods with the use of known growth media. The so obtained plants may be examined for presence of the introduced DNA. Other possibilities of introducing foreign DNA by use of biolistic methods or by protoplast transformation are known as well, and have been described extensively. Once the introduced DNA has integrated itself into the plant cell genome, it generally is stable and is maintained in the progeny of the originally transformed cell as well. Normally it contains a selection marker mediating resistence of the transformed plant cells to a biocide or an antibiotic, such as Kanamycin, G418, bleomycin, hygromycin, methotrexate, glyphosate, streptomycin, sulfonyl urea, gentamycin, or phosphinotricin, and others. The individually chosen marker should therefore allow the selection of transformed cells from cells lacking the introduced DNA. The transformed cells grow normally within the plant. The resulting plants may be grown normally, and interbred with plants containing the same transformed hereditary disposition or other predispositions. The resulting hybrids will have pertinent phenotype characteristics. From the plant cells, seeds may be obtained. Two or more generations should be grown to ensure that the phenotype feature is stably maintained and inherited. Also, seeds should be harvested to verify that the respective phenotype or other features have been maintained. Also, transgenic lines which are homozygous for the new nucleic acid molecules may be determined by usual methods, and their phenotypic behaviour may be studied with respect to a change in fatty acid content, and compared to the behaviour of hemizygous lines.

For the transfer of a resistance marker, a co-transformation is also envisioned, in which the resistance marker is transferred separately. The co-transfer allows the simple subsequent removal of the resistance marker by outbreeding.

Subject matter of the invention are also nucleic acid molecules or fragments thereof which hybridise to a nucleic acid sequence or promoter region in accordance with the invention. The term “hybridisation” as used in the context of this invention refers to a hybridisation under conventional hybridisation conditions, preferably under stringent conditions, such as those described e.g. in Sambrook et al. supra. The molecules which hybridise with the nucleic acid sequences or promoter regions in accordance with the invention comprise also fragments, derivatives, and allelic variants of the nucleic acid sequences and promoter regions. The term “derivative” as used herein means that the sequences of these molecules differ from the sequences in accordance with the invention in one or more positions, and display a high degree of homology with these sequences. Homology refers to a sequence identity of at least 50%, preferably at least 70-80%, and most preferably more than 90%. Deviations may be the result of deletion, addition, substitution, insertion, or recombination.

A person skilled in the art may learn conditions which ensure selective hybridisation from usual laboratory manuals, such as Sambrook et al., supra.

For seed-specific expression of the KCS sequences in accordance with the invention in transgenic plants, any seed-specific regulatory element, particularly promoters, are suitable. Examples are the USP promoter (Bäumlein et al. 1991, Mol. Gen. Genet. 225: 459-467), the hordein promoter (Brandt et al. 1985, Carlsberg Res. Commun. 50: 333-345) as well as the napin promoter, the ACP promoter and the FatB3 and FatB4 promoters which are well known to a person skilled in the art and working in the field of plant molecular biology.

Optionally, the nucleic acid sequences or promoter regions of the invention may be complemented by enhancer sequences or other regulatory sequences. Regulatory sequences include e.g. signal sequences providing transport of the gene product to a particular compartment.

The plants in accordance with the invention are preferably oil seed plants, particularly rapeseed, turnip rape, sun flower, soybean, peanut, coco palm, oil palm, cotton, flax.

Also, the invention relates to a method of providing seed-specific expression of a coding region in plant seeds, comprising the steps of:

a) Generating a nucleic acid sequence in which a promoter region being naturally present in an upstream position to a sequence encoding a protein with KCS activity, is operatively linked with a heterologous coding region,

b) transfer of the nucleic acid sequence from step (a) to plant cells, and

c) regeneration of fully transformed plants, and if desired, propagation of the plants.

As coding region, being expressed under the control of the KCS promoter in accordance with the invention in transgenic plants, any sequence encoding a useful protein is suitable, the protein being useful particularly for food engineering, pharmaceutically or cosmetically, agriculturally, or for the chemical industry. Examples may be proteins playing a role in the biosynthesis of fatty acids and in lipid metabolism, such as desaturases and elongases, acyltransferases, acyl-CoA synthetases, acetyl-CoA carboxylases, thioesterases, as well as glycosyl transferases, sugar transferases and enzymes participating in carbohydrate metabolism. Basically, any interesting protein may be expressed using the KCS promoters in accordance with the invention, so that seeds may be used generally als bioreactors for expression of high quality proteins. Also, the KCS promoters in accordance with the invention are suitable for influencing the structure and color of plant seeds.

The promoter regions in accordance with the invention may also be employed for tissue-specific elimination of undesired gene activities, with antisense and co-suppression techniques being particularly useful.

The invention not only relates to chimeric genes but also to the naturally present combination of KCS promoter and the KCS coding region.

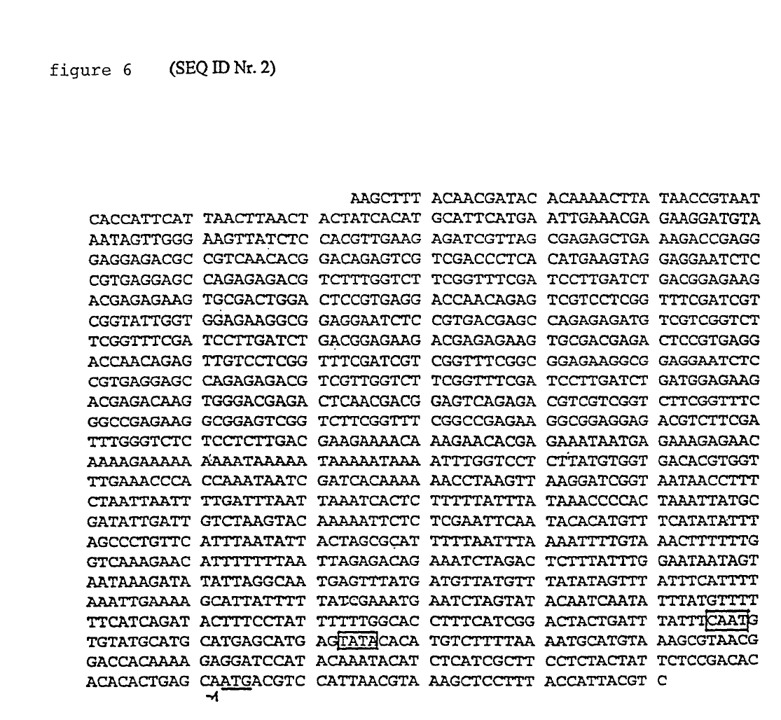

The KCS promoter preferably is a promoter region naturally controlling KCS gene expression in Brassicaceae, most preferably in Brassica napus. Most preferably, the promoter region is a sequence comprised by the sequence depicted in SEQ ID No. 2, the promoter region comprising at least the two promoter elements TATA-box and CAAT-Box (see also highlighted area in FIG. 6).

A further subject matter of the invention is a method of shifting the chain length of fatty acid to longer chain fatty acids in transgenic plants, particularly in oil seed plants, comprising the steps:

a) Generating a nucleic acid sequence in which a promoter region being active in plants and particularly in seed tissue is operatively linked with a nucleic acid sequence encoding rapeseed KCS, and particularly with a coding sequence in accordance with SEQ ID No. 1 or with a sequence encoding a protein in accordance with SEQ ID No. 1 or 3.

b) transfer of the nucleic acid sequence from step (a) to plant cells, and

c) regeneration of fully transformed plants and, if desired, propagation of the plants.

A further subject matter of the invention is a method for increasing the ratio of 22:1 fatty acids to 20:1 fatty acids in transgenic plants, particularly oil seed plants, comprising the steps:

a) Generation of a nucleic acid sequence in which a promoter region being active in plants and particularly in seed tissue is operatively linked with a nucleic acid sequence encoding rapeseed KCS, and particularly with a coding sequence in accordance with SEQ ID No. 1 or with a sequence encoding a protein in accordance with SEQ ID No. 1 or 3.

b) transfer of the nucleic acid sequence from step (a) to plant cells, and

c) regeneration of fully transformed plants and, if desired, propagation of the plants.

The aforementioned methods are not limited to application in transgenic plant cells or plants, but are suitable also for shifting the chain length of fatty acids to longer chain fatty acids, and for increasing the ratio of 22:1 to 20:1 fatty acids in transgenic microorganisms such as fungi, yeasts and bacteria, and algae.

Finally, the invention relates to the use of a nucleic acid sequence encoding a protein with β-ketoacyl-CoA synthase activity for generation of transgenic microorganisms or plant cells with a pattern of polyunsaturated fatty acids being shifted towards longer chain fatty acids compared to the original form.

The term “original form” is used in this context to include the wild-type microorganism and/or the wild-type plant cell and plant, as well as such microorganisms and/or plant cells in which sequences for desaturase and/or further elongase genes have been introduced in addition to a nucleic acid sequence encoding KCS.

Preferably, such nucleic acid sequence is also a nucleic acid sequence encoding a rapeseed KCS, more preferably a nucleic acid sequence comprised by the DNA sequence denoted in SEQ ID No. 1.

It is understood that using the term “nucleic acid sequence in accordance with SEQ ID No. 1 also comprises such nucleic acid sequences being selected from the group constisting of:

a) DNA sequences comprising a nucleic acid sequence encoding the amino acid sequence denoted in SEQ ID No. 1 or 3, or fragments thereof,

b) DNA sequences containing the nucleic acid sequence denoted in SEQ ID No. 1, or parts thereof,

c) DNA sequences comprising a nucleic acid sequence hybridising to a complementary strand of the nucleic acid sequence from a) or b), or parts thereof.

d) DNA sequences comprising a nucleic acid sequence degenerated to a nucleic acid sequence from a), b) or c), or parts of this nucleic acid sequence,

e) DNA sequences being a derivative, analogon or fragment of a nucleic acid sequence from a), b), or d).

The following examples are intended to illustrate the invention.

### EXAMPLES

### Example 1

### Isolation of a Full Length KCS cDNA Clone from Brassica napus

A fragment with a length of approx. 1.0 kb was amplified by PCR from the coding region of the arabidopsis fatty acid elongation gene 1 (FAE1, James et al., supra) using the primers

| | |
|---|---|
| 1: 5′-ATG ACG TCC GTT AAC GTT AAG-3′ | (sense) |
| and | |
| 2: 5′-ATC AGC TCC AGT ATG CGT TC-3′ | (antisense) |

This fragment was used as a heterologous probe for the screening of a rapeseed □-ZAP cDNA library from unripe pods from B. napus cv. Askari (Fulda et al. (1997) Plant Mol. Biol. 33: 911-922). Askari is a HEAR line, containing 55% erucic acid in its seed oil. From approx. 1×10⁶ plaques, 5 positive cDNA clones were isolated. Restriction analysis demonstrated that all 5 clones contained an insert of approx. 1.7 kb in length. Sequence analysis demonstrated that the overlapping regions of the 5′-end as well as of the 3′-end of the cDNAs were identical (approx. 800 bp), but that all cDNAs lacked 8-14 nucleotides, probably including the start codon, at their 5′-end. In order to obtain a full length cDNA clone, a homologous probe was amplified from the longest cDNA clone, using the oligonucleotid primers

| | |
|---|---|
| H1: 5′-CGT TAA CGT AAA GCT CCT TTA C-3′ | (sense) |
| and | |
| H2; 5′-TAG ACC TGA ACG TTC TTG AAT C-3′ | (antisense) |

and was used for further screening experiments with the cDNA library. Since, after two additional screening rounds, still no full cDNA clone was found, a “nested PCR” with template DNA extracted from the cDNA library was used to amplify the 5′-end of the insert. As demonstrated by sequence analysis of the amplified fragments, this approach did also not lead to the detection of a full length clone in the library. Therefore, an inverse PCR (Ochman et al. (1988) Genetics 120: 621-623) was used to clone the missing 5′-end with genomic DNA from the Askari rapeseed line as a template. Two specific primers

| | |
|---|---|
| IP1: 5′-TGA CGT AAT GGT AAA GGA GC-3′ | (sense) |
| and | |
| 1P3: 5′-TTC AAG CTC CGA AGC AAC-3′ | (antisense) |

were constructed, corresponding to the 5′-end of the cloned cDNA, but in reverse directions. For digestion of the genomic DNA, the restriction enzyme HindIII was employed, since there was a HindIII restriction site located downstream of the primer IP3, however, no HindIII-site was located in the region between the primers. After digestion and ligation of the genomic DNA, the orientation of the primers was reversed to allow the PCR to take place. By the use of DNA polymerases with proof reading capacity, such as pfu from Stratagene, a 1.5 kb fragment could be amplified. The PCR fragment was cloned and sequenced. The DNA sequences from three independent clones were identical, and contained the missing 5′-end (AGCAATGACGTC, with the assumed start codon being underlined) of the cDNA.

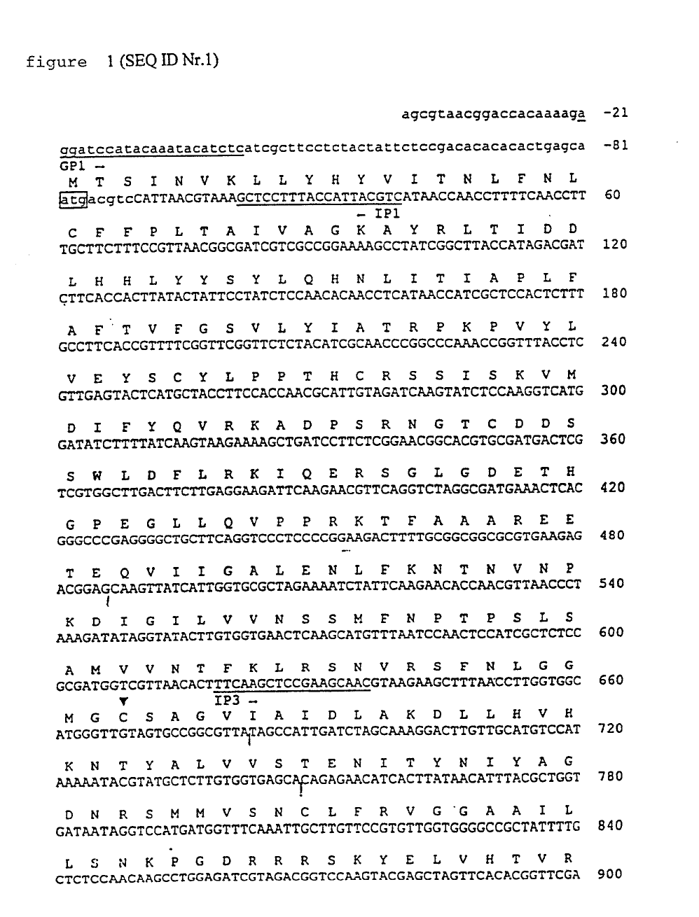

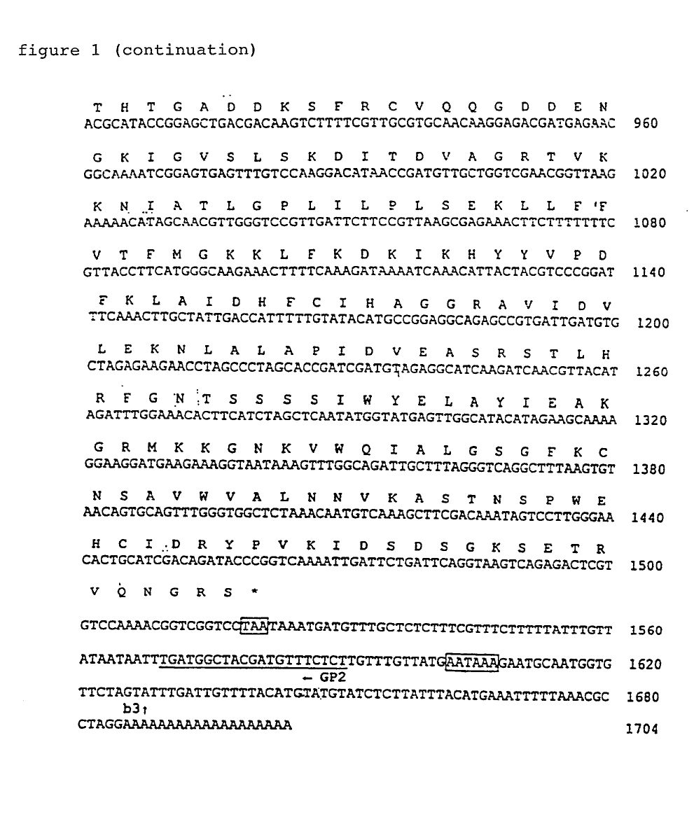

The complete nucleotide sequence and the deduced amino acid sequence of the KCS cDNA from B. napus cv. Askari are depicted in FIG. 1 (SEQ ID Nr. 1). The primers used for the inverse PCR are underlined in FIG. 1. Underlined as well are the other primers that were used for the amplification of genomic DNA from B. napus cv. Drakkar and line RS306 (see Example 2). Forward and reverse primers are indicated by horizontal arrows. The assumed start codon and stop codon and the polyadenylation sequence are framed. The polyA signal of clone #b3 is indicated by a vertical arrow. The assumed active site Cys223 is indicated by a filled triangle.

The open reading frame (ORF) has a length of 1521 bp and encodes a polypeptide of 506 amino acids (plus stop codon) having a predicted molecular weight of 56.4 kDa, and an isoelectric point value of 9.18.

Northern blot analyses were performed to determine the expression pattern of the KCS gene in B. napus. For this purpose, total RNA from leaves and immature embryos in various developmental stages was isolated from Askari rapeseed plants by standard methods, and was hybridised with a B. napus KCS-cDNA-specific probe. As expected, a 1.7 kb transcript was detected in developing embryos only, but not in leaves. In embryos, this transcript was clearly detectable 16 days after pollination, then its concentration increased gradually and peaked at approx. 30 days after pollination, and again decreased slightly until the 40^{th} day after pollination. These northern blot data demonstrate clearly that expression of the KCS gene in wild-type rapeseed plants is regulated temporally as well as spatially.

### Example 2

### Isolation of Genomic KCS Clones from B. napus

For isolation of genomic KCS clones from the B. napus line RS306, a HEAR line, and from B. napus cv. Drakkar, a LEAR variety (22:1<1%), the primers

| | |
|---|---|
| GP1:5′-AGG ATC CAT ACA AAT ACA TCT C-3′ | (sense) |
| and | |
| GP2:5′-AGA GAA ACA TCG TAG CCA TCA-3′ | (antisense) |

were used which were derived from the 5′- and 3′-UTRs of the cDNA shown in FIG. 1. Both genomic KCS sequences from RS306 and from Drakkar contained an ORF of 1521 bp (identical to the cDNA ORF, see example 1), which means that the rapeseed KCS gene does not contain any introns. The deduced proteins contained 506 amino acid residues with a molecular weight of 56.46 kDa and 56.44 kDa, respectively, and a pI of 9.18 and 9.23, respectively. Compared to the cDNA in FIG. 1, the deduced amino acid sequence of the genomic KCS clone from RS306 contained four amino acid exchanges at positions 286 (Gly286Arg), 323 (Ile323Thr), 395 (Arg395Lys), and 406 (Ala406Gly), whereas the genomic sequence from Drakkar contained only one exchange at position 282 (Ser282Phe) compared to the Askari cDNA.

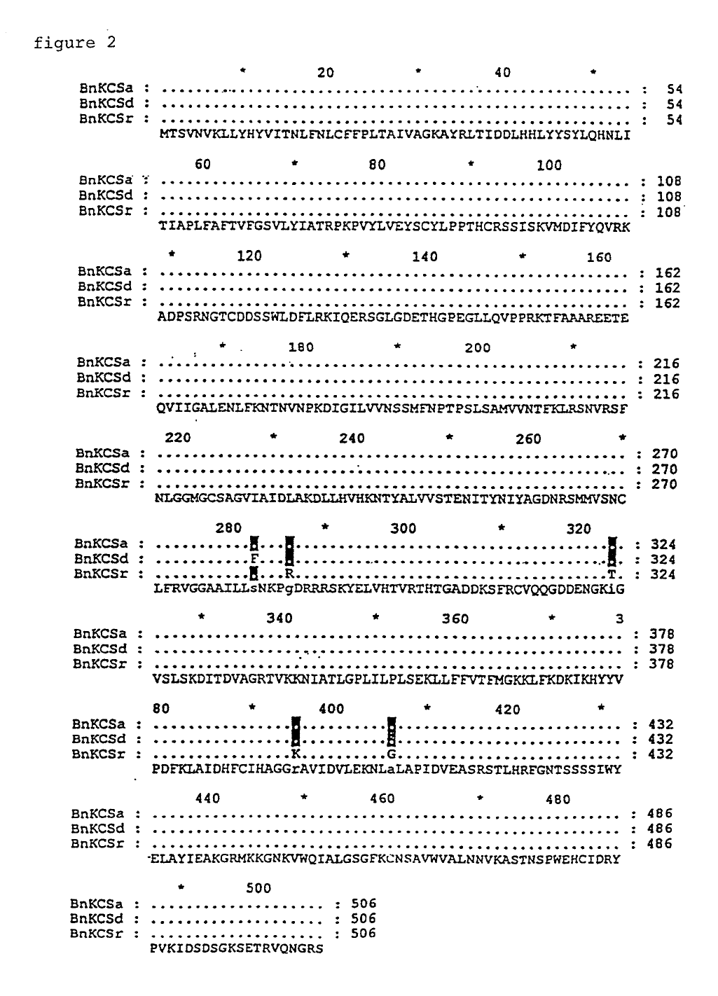

These amino acid sequence differences are additionally illustrated in FIG. 2. BnKCSa=KCS cDNA from B. napus cv. Askari, BnKCSd=genomic KCS clone from B. napus cv. Drakkar, and BnKCSr=genomic KCS clone from B. napus RS306.

It is presently assumed that the mutation in position 282 (Ser282Phe) results in a catalytically inactive KCS protein, and therefore causes the LEAR phenotype.

Various hints support the hypothesis that residue Ser282 is of essential importance for the KCS activity of the wild-type protein, the role of the serine residue being structural rather than catalytical.

Finally, it is noted that the sequence depicted in SEQ ID No. 1 differs from the sequence published by Clemens and Kunst (1997, vide supra) with respect to amino acid 307.

### Example 3

### Expression of KCS From B. napus in Transgenic B. napus Plants

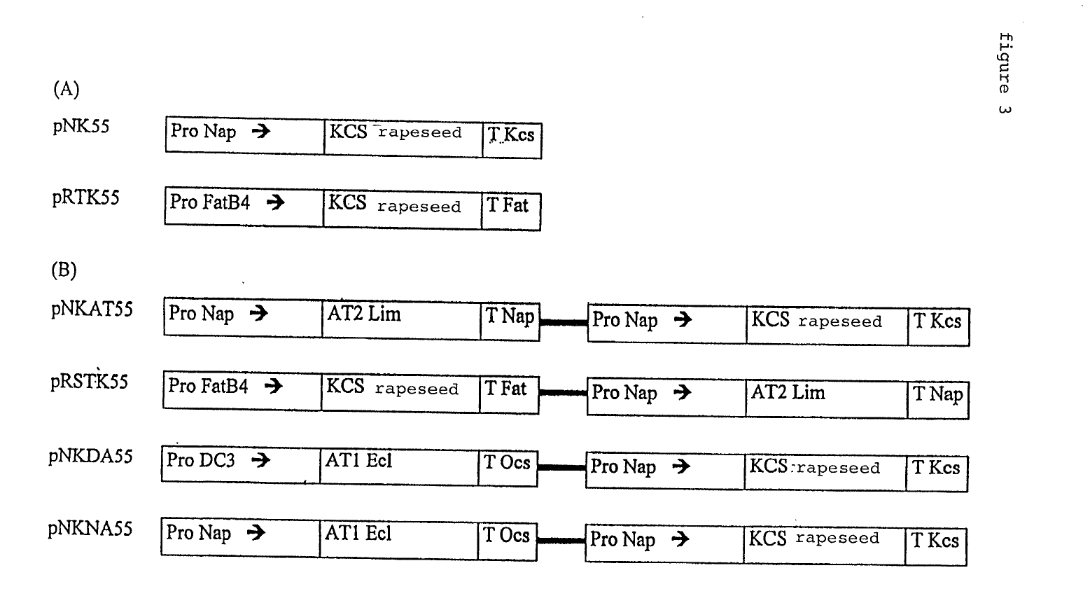

For the expression of KCS from B. napus cv. Askari in transgenic plants, various plasmid constructs were generated, which are illustrated in FIG. 3. For the construction of KCS gene fusions, an EcoRI restriction site (underlined in Y1) was introduced at the 5′-end with the help of the primer

Y1: 5′-GGA ATT CAA ACA AAT GAC GTC CGT TAA CGT AAA GCT-3′ (sense)

A 522 bp fragment containing the 509 bp cDNA coding region and the 13 bp 5′-UTR was amplified by PCR using the primer pair Y1/Y2, and purified in an agarose gel; primer Y2 had the sequence

Y2: 5′-TCT AGC GCA CCA ATG ATA AC-3′ (antisense)

The fragment was cloned into the vector pGEM-T (Promega) and sequenced; the resulting vector was termed pNK51. The last 1.3 kb of the cDNA were cut out with ApaI, and ligated into pNK51 which was also digested with ApaI; the resulting plasmid was termed pNK52. For the fusion of the cDNA with the gNA Napin gene promoter from B. napus (Scofield and Crouch (1987) J. Biol. Chem. 262: 12202-12208), a 2.2 kb PstI/HindIII fragment with the Napin promoter was excised from pGEM-Nap, and was ligated into the respective restriction sites of the vector pBluescript KS⁻ (Stratagene); the resulting vector was termed pNK53. A 1.7 kb fragment with the cDNA coding region and its 3′-polyA signal was excised from pNK52 with SpeI/BsmI, and its ends filled up with Klenow. The resulting fragment with blunt ends was introduced downstream of the Napin promoter into pNK53, which had previously been digested with HindIII and treated with Klenow, in order to obtain pNK54. A 3.9 kb fragment with the chimeric KCS gene was then cloned into the SpeI/SalI-digested binary vector pRE1 to obtain pNK55. pRE1 contains a chimeric neomycin phosphotransferase gene as selection marker, but any other vector suitable for plant transformation, and particularly any other binary vector, may be used as well. For a tandem construct, a 3.3 kb SpeI-fragment containing a chimeric Limnanthes douglasii LPAAT gene was excised from pRESS (Weier et al. (1997) Fett/Lipid 99: 160-165), and then ligated into SpeI-digested pNK55, generating the construct pNKAT55.

For the construction of fusions of the KCS coding regions with the acyl-ACP thioesterase gene FatB₄ promoter from Cuphea lanceolata, a 1.7 kb EcoRI/XhoI-BCS fragment from pNK54 was inserted into a suitable vector between the FatB₄ promoter and its termination signal. A 5.2 kb fragment containing the chimeric KCS gene was excised with SfiI, its ends filled up with Klenow, and was subsequently cloned into pRE1 and pRESS (Weier et al. supra) digested with SmaI, generating the vectors pRTK55 and pRSTK55, respectively.

For generation of KCS tandem constructs with a plsB gene encoding the sn-glycerol-3-phosphate acyltransferase from E. coli (Lightner et al. (1980) J. Biol. Chem. 19: 9413-9420; Lightner et al. (1983) J. Biol. Chem. 258: 10856-10861), two restriction sites, KpnI (underlined in AT1) and MscI (underlined in AT2), were introduced using the two primers

| | |
|---|---|
| AT1: 5′-CGG GGT ACC GGC GGC CGC TCT | (sense) |

AG-3′ and

| | |
|---|---|
| AT2: 5′-CGT GGC CAG CCG GCC ATG GTA ATT | (antisense) |

GTA AAT G-3′

A 280 bp PCR fragment containing a seed-specific DC3 promoter from carrot (Seffens et al. (1990) Dev. Genet. 11: 65-76) and a leader sequence Ω from tobacco mosaic virus (Gallie et al. (1987) Nucl. Acids. Res. 15: 3257-3273) was cloned into pGEM-T (Promega) to obtain pGEM-DC3. A 3.0 kb HindIII/SmaI fragment containing the 2.5 kb plsB-coding region, the 0.25 kb Ocs-termination sequence, and the 0.25 kb 5′-UTR were excised from pHAMPL4 (Wolter et al. (1992) EMBO J. 11: 4685-4692), and cloned into HindIII/HincII-digested pBluescript KS⁻. The 0.25 kb 5′-UTR was removed by digestion with KpnI/MscI, and a 300 bp DC3Ω fragment from pGEM-DC3 was then inserted to obtain pDC3-1AT. The resulting chimeric gene (3.1 kb) was then ligated into the SpeI-digested plant expression vector pNK55 to obtain pNKDA55. For the plsB gene fusion with the Napin promoter, a 2.8 kb NcoI/NotI-fragment containing the plsB-coding region and the Ocs terminator from pDC3-1AT, were ligated into the vector pGEM-T (Promega) which had been double digested with the same enzymes. The resulting plasmid pGEM-1AT was digested with ApaI/NotI, Klenow-treated, and the blunt end fragment was inserted downstream of the Napin promoter into HindIII-digested and Klenow-treated pNK53. The resulting chimeric gene (5.0 kb) was excised with SpeI and ligated into SpeI-digested vector pNK55 to obtain pNKNA55.

As mentioned, the generated plant expression constructs are schematically depicted in FIG. 3. ProNap=Napin promoter, ProFatB4=FatB4 promoter, ProDC3=DC3 promoter, AT2Lim=Limnanthes LPAAT cDNA, KCSRaps=rapeseed KCS cDNA, AT1Ecl=E. coli GPAT gene, TKcs, T Nap, and T Ocs=polyA signals from KCS, FatB4, Napin (nap) and Agrobacterium octopine synthase (Ocs), respectively.

The first group of the constructs used for generation of transgenic plants therefore consists of single constructs in which the KCS cDNA is under the control of a seed-specific promoter of either the Napin gene gNA from B. napus (Scofield et al., supra), or the acyl-ACP thioesterase gene FatB₄ from Cuphea lanceolata.

The second group of constructs consists of double or tandem constructs containing a chimeric KCS gene in combination with the coding sequence of either the sn-1-acyl-glycerol-3-phosphate acyltransferase from L. douglasii (LPAAT) (Hanke et al. (1995) Eur. J. Biochem. 232: 806-810), or the sn-glycerol-3-phosphate acyltransferase (GPAT) from E. coli under the control of either the Napin promoter or the FatB₄ promoter, or the DC3 promoter from carrot (Seffens et al., supra) plus a 5′-leader sequence (Ω) from tobacco mosaic virus (Gallie et al., supra)(see FIG. 3, B). These constructs were introduced into suitable binary vectors and transferred to Agrobacterium tumefaciens (strains GV3101/pMP90, Koncz and Schell (1986) Mol. Gen. Genet. 204: 383-396, and C58ATHV/pEH101, Hood et al. (1986) J. Bacteriol. 168: 1291-1301) for rapeseed transformation. The single constructs were transferred to the LEAR variety Drakkar, and the double constructs were transferred to the HEAR line RS306.

The transformation was performed using co-cultivation of hypokotyl explants and transformed agrobacteria, and the transgenic sprouts were selected on a kanamycin-containing medium according to standard methods (see De Block et al. (1989) Plant Physiol. 91: 694-701). Transgenic plants were screened for presence of the desired genes by southern blotting using suitable probes.

Mature seeds were collected from transgenic self-pollinated LEAR-Drakkar plants containing the Napin-KCS or FatB₄-KCS constructs, and pooled T2-seeds were used for determination of the fatty acid composition of the seed oils. The collected data are summarised in Table 1 below. Table 1 contains the fatty acid composition of pooled T2-seeds from transgenic LEAR-Drakkar-plants and from Drakkar control plants (ck). T-NK represents T2-seeds from Napin-KCS plants, whereas T-RTK identifies T2-seeds from FatB₄-KCS plants.

TABLE 1

| | Percent fatty acids per weight | | | | | | | | |
|---|---|---|---|---|---|---|---|---|---|
| Plant | 16:0 | 18:0 | 18:1 | 18:2 | 18:3 | 20:1 | 22:1 | 24:1 | VLCFA |
| Drak(ck) | 3.0 | 1.9 | 66.7 | 15.2 | 8.5 | 1.9 | 0.1 | 0.3 | 2.3 |
| T-NK-4 | 3.1 | 2.2 | 65.1 | 9.8 | 4.6 | 7.3 | 5.6 | 0.4 | 13.3 |
| T-NK-5 | 3.5 | 2.9 | 66.1 | 9.7 | 4.5 | 8.3 | 3.4 | 0.3 | 12.0 |
| T-NK-10 | 3.1 | 2.5 | 65.8 | 9.7 | 4.4 | 8.0 | 4.1 | 0.4 | 12.5 |
| T-NK-11 | 3.5 | 2.4 | 63.9 | 10.2 | 4.6 | 9.3 | 3.9 | 0.4 | 13.6 |
| T-NK-13 | 3.3 | 2.3 | 61.7 | 9.3 | 4.4 | 11.1 | 5.9 | 0.5 | 17.5 |
| T-NK-14 | 3.3 | 2.7 | 69.9 | 11.6 | 4.6 | 4.2 | 1.7 | 0.3 | 6.2 |
| T-NK-15 | 2.9 | 1.9 | 54.7 | 8.6 | 5.5 | 15.1 | 9.1 | 0.5 | 24.7 |
| T-NK-16 | 3.4 | 2.2 | 67.3 | 9.6 | 4.9 | 8.5 | 2.2 | 0.4 | 11.1 |
| T-NK-18 | 3.4 | 2.5 | 67.6 | 9.1 | 4.7 | 8.7 | 2.3 | 0.4 | 11.4 |
| T-NK-20 | 3.1 | 3.5 | 47.2 | 6.6 | 3.5 | 14.8 | 15.5 | 0.7 | 31.0 |
| T-NK-21 | 3.5 | 2.6 | 67.2 | 9.9 | 3.9 | 7.8 | 2.5 | 0.3 | 10.6 |
| T-NK-24 | 3.3 | 2.3 | 73.4 | 9.1 | 4.2 | 4.4 | 1.3 | 00.3 | 6.0 |
| T-NK-26 | 3.1 | 2.3 | 61.8 | 12.5 | 6.7 | 9.0 | 2.1 | 0.2 | 11.3 |
| T-NK-27 | 4.1 | 1.8 | 58.6 | 18.6 | 8.0 | 4.9 | 1.6 | 0.5 | 7.0 |
| T-NK-30 | 2.9 | 1.8 | 58.2 | 11.4 | 6.5 | 12.6 | 4.1 | 0.4 | 17.1 |
| T-NK-32 | 2.9 | 2.1 | 55.0 | 10.9 | 6.6 | 14.2 | 5.6 | 0.5 | 20.3 |
| T-NK-33 | 3.5 | 2.5 | 60.6 | 11.2 | 7.0 | 7.2 | 5.1 | 0.5 | 12.8 |
| T-NK-34 | 3.3 | 1.6 | 60.3 | 15.4 | 8.0 | 7.2 | 1.6 | 0.5 | 9.3 |
| T-NK-35 | 2.6 | 3.2 | 55.4 | 6.2 | 4.1 | 16.4 | 6.7 | 0.6 | 23.7 |
| T-NK-38 | 2.9 | 2.6 | 69.5 | 7.0 | 4.3 | 8.5 | 3.0 | 0.4 | 11.9 |
| T-NK-40 | 3.1 | 1.8 | 65.5 | 11.1 | 7.3 | 6.4 | 2.3 | 0.4 | 9.1 |
| T-NK-41 | 3.2 | 2.7 | 59.6 | 9.7 | 5.7 | 11.7 | 4.8 | 0.5 | 17.0 |
| T-NK-42 | 3.6 | 2.0 | 60.4 | 14.4 | 8.3 | 6.9 | 1.8 | 0.4 | 9.1 |
| T-NK-43 | 3.4 | 1.4 | 59.8 | 14.7 | 10.3 | 7.0 | 1.3 | 0.4 | 8.7 |
| T-NK-47 | 3.2 | 1.8 | 59.9 | 14.7 | 8.7 | 7.6 | 1.6 | 0.4 | 9.6 |
| T-NK-49 | 0.3 | 1.8 | 54.1 | 10.8 | 7.3 | 12.8 | 7.5 | 0.7 | 21.0 |
| T-NK-50 | 2.8 | 2.4 | 64.1 | 9.1 | 5.4 | 9.4 | 2.9 | 0.5 | 12.8 |
| T-NK-65 | 2.9 | 2.2 | 57.1 | 9.5 | 6.0 | 14.6 | 5.4 | 0.5 | 20.5 |
| T-NK-71 | 3.7 | 2.6 | 66.3 | 11.4 | 8.0 | 3.6 | 1.7 | 0.4 | 5.7 |
| T-NK-82 | 3.7 | 2.7 | 61.5 | 10.3 | 5.9 | 11.1 | 4.2 | 0.2 | 15.5 |
| T-NK-85 | 3.9 | 2.3 | 56.8 | 14.9 | 8.6 | 8.8 | 2.7 | 0.4 | 11.9 |
| T-RTK-2 | 3.6 | 2.6 | 67.9 | 10.6 | 4.7 | 7.5 | 1.5 | 0.4 | 9.4 |
| T-RTK-94 | 3.1 | 2.2 | 64.2 | 9.9 | 5.7 | 8.5 | 1.6 | 0.4 | 10.5 |

The seed oil of wild-type plants contained less than 3% VLCFA, whereas up to 18% 20:1^{Δ11} and up to 16% 20:1¹³ could be detected in the fatty acid composition of transgenic seed oils. The 24:1 content in transgenic seed oils reached a maximum of 0.9%. Whereas 22 out of 44 Napin KCS plants had high VLCFA concentrations in the range of 11 to 31%, only 2 out of 70 FatB4 KCS plants reached a content of approx. 10% VLCFAs. Generally, the increase in VLCFA was accompanied by a decrease in the content of unsaturated C18-fatty acids, whereas the 16:0 and 18:0 content was changed only minimally. The differences in VLCFA amounts in the seed oils of independent transformants may be due to different KCS expression rates. In summary, the results demonstrate that the B. napus CDNA in fact encodes a β-ketoacyl-CoA transferase which catalyses both elongation steps from 18:1 to 22:1, but which is only minimally active with 22:1-CoA as a substrate. The introduction of only one KCS as the single condensing enzyme resulted in significant amounts of VLCFAs, which means that the other three enzymes being required for VLCFA synthesis, the above mentioned two reductases and the dehydratase, have to be present functionally in the microsomal elongation system of Drakkar plants.

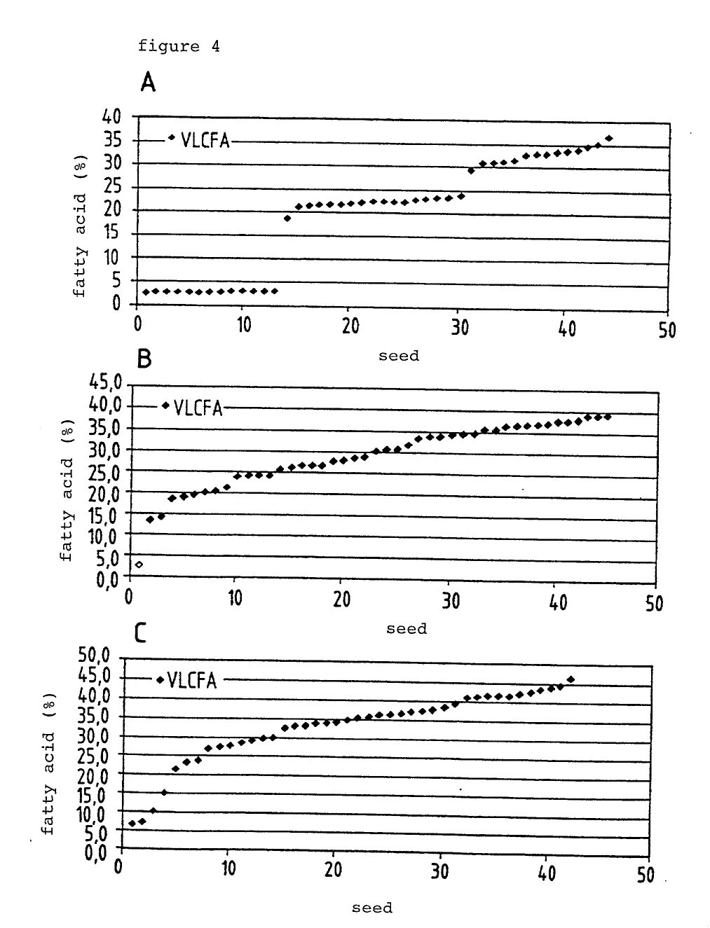

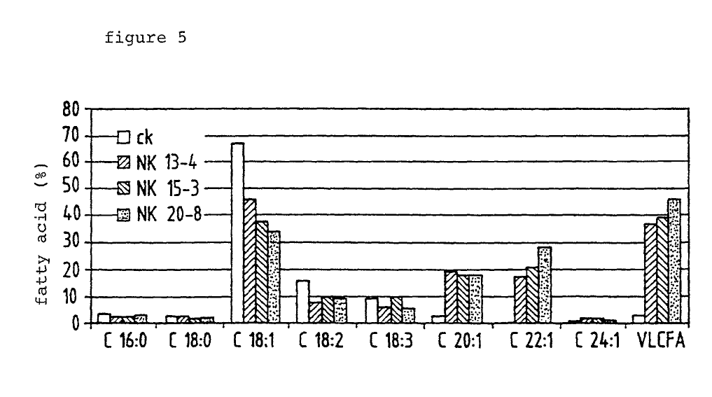

Since T2 seeds split up for each T-DNA insert, it could be assumed that individual seeds that were homozygous for the T-DNA insert had a higher VLCFA content. Therefore, individual cotyledones from T2 seeds from three transgenic plants (T-NK-13,-15, and -20) were used for further analyses of the fatty acid composition. The results are shown in FIG. 4, depicting the distribution of the VLCFA content in individual T2 seeds from transgenic LEAR-Drakkar plants. (A) VLCFA content of 44 individual seeds from plant T-NK-13, (B) VLCFA content of 45 individual seeds from plant T-NK-15, and (C) VLCFA content of 42 individual seeds from plant T-NK-20. As expected and due to gene dose effects, certain individual seeds had higher VLCFA contents compared to those contents that had been measured in pooled seed oil fractions. In T2 seeds of transformant T-NK-13, 12 out ot 44 seeds demonstrated a VLCFA content that was almost twice as high as the VLCFA content of the pooled T2 seeds, whereas 13 seeds displayed the fatty acid pattern of the wild-type. These data show that a T-DNA locus was present in the primary transformants of T-NK-13. On the other hand, the analysis of transformants T-NK-15 and T-NK-20 suggested that at least three active copies of the transgene were present in these transformants, since only one out of 45 seeds in T-NK-15, and not a single seed from 42 T-NK-20 transformants had a LEAR genotype. In individual seeds from T-NK-20, up to 28% 22:1^{Δ13} and 45% VLCFA could be detected. Furthermore, the seed pol analysis showed that the 22:1/20:1 ratio was highly depending on the activity of the introduced KCS enzyme, which was reflected in the total VLCFA content of the seed oils. 22:1/20:1 ratios of>1 were only observed, when VLCFA contents were above 39% (see FIG. 4 and FIG. 5). FIG. 5 shows the fatty acid composition of individual T2 seeds from transgenic LEAR-Drakkar plants compared to control plants (ck); NK13-4=seeds from a T-NK-13 plant, NK15-3=seeds from a T-NK-15 plant, NK20-3=seeds from a T-NK-20 plant.

In order to increase the erucic acid content in triacylglycerides on the basis of HEAR phenotypes, not only the 22:1 content in the CoA seed pool has to be increased, but also, the 22:1 content has to be channeled into the oil and the sink for fatty acid deposits. For this purpose, the above described expression vectors were constructed in which the rapeseed KCS is present under the control of either the Napin promoter, or the FatB4 promoter, or the DC3 promoter, and in combination with either LPAAT (from L. douglasii) in order to manipulate the channeling of 22:1 into the sn-2 position of the seed oil, or with GPAT (from E. coli) in order to increase the sink-capacity for fatty acid deposits. The constructs NKAT (napin-KCS-napin-LPAAT), RSTK (FatB4-KCS-napin-LPAAT), NKDA (napin-KCS-DC3-GPAT), and NKNA (napin-KCS-napin-GPAT) were transferred to the HEAR line RS306. Pooled T2 seeds from transgenic RS306 plants were analysed for their fatty acid composition, and the results are summarised in Table 2. RS306 (ck) identifies the seed oil from RS306 control plants which were transformed with the empty vector pRE1. T-NKAT represents T2 seeds from NKAT plants, T-RSTK represents T2 seeds from RSTK plants, T-NKDA represents T2 seeds from NKDA plants, and T-NKNA represents T2 plants from NKNA plants.

TABLE 2

| | Percent fatty acids per weight | | | | | | | | TAG species | |
|---|---|---|---|---|---|---|---|---|---|---|
| Plant | C16:0 | C18:0 | C18:1 | C18:2 | C18:3 | C20:1 | C22:1 | C24:1 | EiEE | EEE |
| RS306 (ck) | 2.5 | 1.3 | 15.7 | 10.8 | 4.1 | 6.5 | 53.7 | 1.9 | — | — |
| T-NKAT-1 | 2.4 | 1.1 | 13.3 | 11.5 | 5.1 | 7.0 | 55.0 | 1.6 | 2.8 | 2.9 |
| T-NKAT-5 | 2.3 | 1.3 | 13.0 | 10.1 | 4.1 | 6.3 | 56.7 | 1.6 | 3.0 | 3.7 |
| T-NKAT-6 | 2.1 | 1.0 | 11.8 | 10.4 | 5.3 | 8.2 | 55.5 | 1.5 | 4.3 | 4.1 |
| T-NKAT-7 | 2.0 | 1.9 | 12.7 | 10.8 | 4.4 | 8.3 | 55.3 | 1.5 | 4.2 | 4.1 |
| T-NKAT-14 | 2.1 | 1.9 | 11.9 | 11.6 | 5.4 | 6.1 | 55.9 | 1.7 | 3.8 | 4.3 |
| T-RSTK-13 | 2.1 | 1.8 | 11.1 | 11.1 | 6.4 | 5.9 | 56.7 | 1.9 | 4.3 | 5.6 |
| T-RSTK-15 | 2.0 | 1.0 | 14.5 | 10.7 | 4.1 | 7.0 | 55.3 | 1.6 | 3.5 | 2.9 |
| T-NKDA-5 | 1.9 | 1.2 | 12.1 | 11.0 | 4.9 | 6.4 | 58.2 | 2.0 | — | — |
| T-NKDA-7 | 2.3 | 1.2 | 11.4 | 10.0 | 5.1 | 5.2 | 59.6 | 2.3 | — | — |
| T-NKDA-15 | 1.9 | 1.2 | 11.1 | 11.2 | 4.5 | 5.8 | 58.7 | 2.1 | — | — |
| T-NKDA-16 | 1.8 | 1.2 | 12.5 | 11.3 | 4.9 | 5.3 | 58.0 | 1.9 | — | — |
| T-NKDA-9 | 2.1 | 1.4 | 11.7 | 11.2 | 4.5 | 5.7 | 57.0 | 2.7 | — | — |
| T-NKDA-4 | 1.9 | 0.9 | 10.0 | 13.5 | 5.9 | 5.1 | 57.6 | 1.8 | — | — |
| T-NKNA-3 | 1.6 | 1.0 | 12.8 | 11.0 | 5.0 | 5.4 | 57.7 | 2.0 | — | — |
| T-NKNA-15 | 2.0 | 1.3 | 10.7 | 12.4 | 5.4 | 4.8 | 56.3 | 2.4 | — | — |
| T-NKNA-20 | 1.8 | 1.1 | 16.1 | 8.7 | 4.3 | 8.1 | 56.4 | 1.7 | — | — |

In Table 2, “EiEE” represents triacylglyceride with a eicosenoic acid residue (20:1) and two erucic acid residues (22: 1). “EEE” represents trierucin, which is triacylglyceride with three erucic acid residues.

In T2 seed oils, a small increase in the 22:1 content in the range of 2.6 to 5.9% could be observed compared to RS306 control plants. The transgenic plants accumulated 2.9-5.6% trierucin (EEE) in their seed oil. The percentual fraction of 22:1 at position sn-2 in triacylglyceride (TAG) reached 31.7-37.5% in the transgenic seed oils, whereas in the control seeds the percentual fraction was less than 1% of the sn-2 fatty acids. These results demonstrate that the introduced KCS and LPAAT genes were expressed operatively in the transgenic plants. Furthermore, the data shown in Table 2 suggest that in HEAR plants a 22:1 content of max. 60-65% may be obtained.

### Example 4

### Analysis of the Rapeseed KCS Promoter

As described above in example 1, an inverse PCR was performed to complete the region of the start codon of the KCS cDNA, and various 5′-flanking sequences from the KCS coding region with a length of ˜1.5 kb were isolated from the genomic DNA from three different rapeseed varieties (B. napus cv. Askari, Drakkar, and RS line 306). Sequence analysis showed that the promoter sequences of these clones were identical, therefore, a promoter which had been isolated from Askari was chosen for further analysis.

FIG. 6 shows the sequence of the KCS promoter from rapeseed (SEQ ID Nr. 2); the sequence comprises 1468 bases in total. The 5′-end of the shown sequence corresponds to the nucleotide −1429 of the KCS gene, whereas at the 3′-end, the shown sequence comprises codons 1 (methionine) to 13 (valine) of the KCS coding sequence. The ATG start codon, the CAAT box, and the TATA box are plotted.

No similarities were observed between the KCS promoter region and any other promoter sequences available from the data bases.

The KCS promoter not only shows AT-rich elements (19 elements with a length between 6 and 19 bp in the region from −1 and −471) which are typical for seed-specific promoters, but also various other motifs in the region −99 to −137, suggesting a tissue-specific regulation. An RY repeat (CATGCATG) is present between the CAAT box and the TATA box, and an E box is present next to the TATA box.

For analysis of the functional and tissue-specific expression in transgenic rapeseed plants, a 1.5 kb promoter region from the KCS gene was fused with the reporter gene uidA encoding β-glucuronidase (GUS) (Jefferson et al. (1987) Plant. Mol. Biol. Rep. 5: 387-405; Jefferson et al. (1989) EMBO J. 6: 3901-3907) in the binary vector pBI101.2 (Clontech, Calif.; Jefferson et al., supra). For this purpose, a PCR was performed using the following primers:

| | |
|---|---|
| IP6: 5′-CTC TCG AAT TCA ATA CAC ATG-3′ | (sense) |
| and | |
| IP8: 5′-TCC CCC GGG TGC TCA GTG TGT GTG | (antisense) |

TCG-3′

with IP6 overlapping the promoter region, and the reverse primer IP8 containing an introduced SmaI site (underlined) for cloning purposes. A 470 bp PCR fragment was ligated into the vector pGEM-T (Promega) and sequenced. The PCR fragment was excised with the restriction enzymes EcoRI and NcoI and ligated into the 3′-end of the promoter that had been digested with the same enzymes. Finally, a 1.5 kb promoter fragment was excised with the restriction enzymes HindIII and SmaI, and inserted into pBI101.2 in front of the GUS coding region. The resulting construct was termed pBnKCS-Prom.

The promoter/GUS construct was transferred to B. napus RS306, and immature seeds in various developmental stages as well as other tissues from transgenic plants and control plants were used for GUS analysis. The histochemical GUS staining demonstrated GUS activity in developing seeds from transgenic plants only, but not in roots, stalks, leaves, buds and flowers from transgenic plants, and also not in organs of the control plants. In transgenic seeds, the GUS expression became visible at day 16 after pollination and increased up to day 30 after pollination, correlating with the expression pattern of the native KCS gene. The histochemical results were verified by quantitative chemiluminescence analysis. In transgenic seeds harvested at days 25 and 30 after pollination, GUS activities of up to 180 and 324 μmol/min/mg protein, respectively, could be measured. These data demonstrate that the promoter region depicted in FIG. 6 represents a novel very active seed-specific promoter with high expression rate in transgenic rapeseed plants.

### Example 5

### Expression of KCS From B. napus in Yeast

In order to compare function and activity of the KCS coded by the various isolated KCS genes from Askari, Drakkar and the RS line 306, the genes were expressed in the strain INVSC1 from Saccharomyces cerevisiae (Invitrogen) under the control of a galactose-inducable GAL1 promoter.

For this purpose, the various isolated KCS sequences were fused with the GAL1 promoter in the yeast expression vector pYES2 (Invitrogen, Calif.). A 1.7 kb BnKCSa fragment from the cDNA library from B. napus cv. Askari was excised with the restriction enzymes EcoRI and XhoI and inserted into the vector pYES2 cut with the same enzymes, generating the vector pYES-BnKCSa. For the two other yeast expression constructs, a 0.8 kb HindIII-fragment from BnKCSa was substituted with the fragment from BnKCSd, being the genomic DNA sequence from B. napus cv. Drakkar. The resulting 1.7 kb chimeric BnKCSd gene was inserted into the EcoRI/XhoI digested vector pYES2, generating the vector pYES-BnKCSd. For the last construct, which was the yeast expression vector containing the genomic KCS sequence from line RS306, a 0.9 kb ClaI/EcoRV fragment from BnKCSa was substituted by the fragment from BnKCSr (KCS sequence from line RS306). The plasmid DNAs were isolated from E. coli strain SCS110 (Stratagene). The resulting chimeric gene BnKCSr (1.7 kb) was inserted into EcoRI/XhoI digested pYES2 to obtain pYES-BnKCSr.

INVSC1 cells containing the plasmid pYES2 without insert were used as wild-type control. The fatty acid composition of the yeast cells were determined by gas liquid phase chromatography (GLC), and the components of the VLCFAs were identified by GLC-MS (GLC mass spectroscopy). Significant amounts of VLCFAs were found in the transgenic yeast cells with the KCS sequence from Askari, whereas the transgenic yeast cells expressing the KCS sequences from Drakkar or RS line showed fatty acid compositions similar to those of the control cells (see also Table 3). In cells with the Askari KCS sequence, up to 41% VLCFAs in the fatty extracts were detected, in which 22:1 fatty acids with double bonds were predominant in position Δ15 or Δ13, but saturated and monounsaturated fatty acids with more than 22 carbons in noticable amounts could also be detected. These data show that the KCS gene from Askari and not the KCS genes from Drakkar or from the RS306 line was operatively expressed in yeast, and was cooperating effectively with the components of the yeast elongase complex. Furthermore these data show, that the KCS expressed in yeast has a relatively broad acyl-CoA specificity.

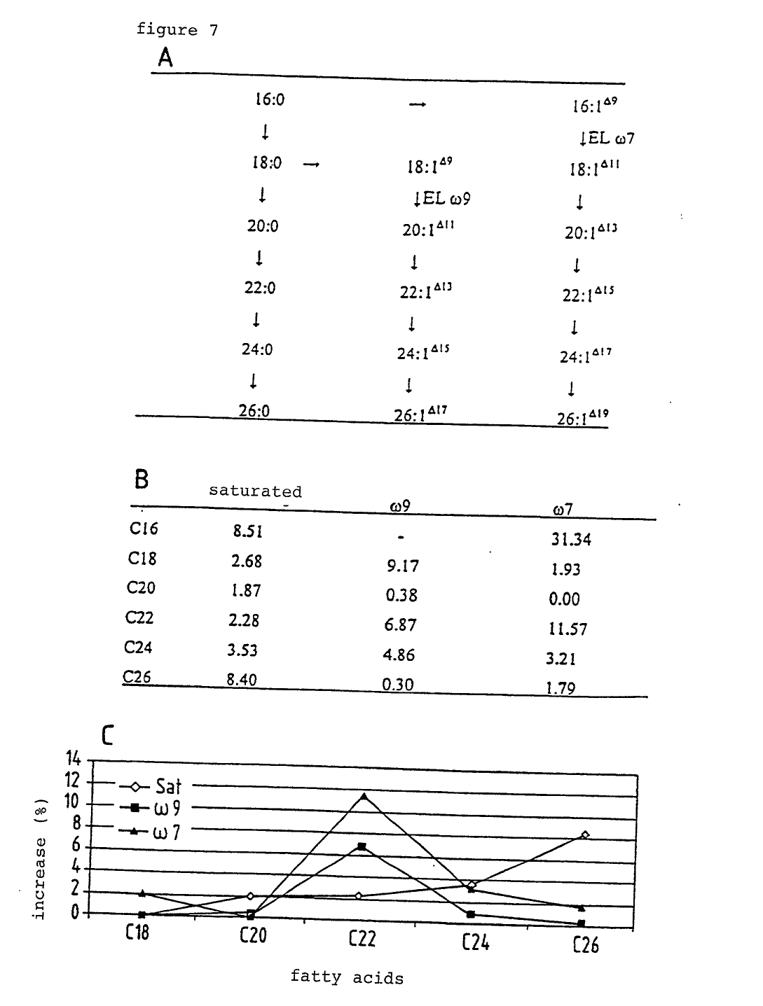

As shown in FIG. 7A, the KCS not only uses 18:1_{Δ9} but also 16:1^{Δ9} acyl groups as a substrate. The KCS seems to utilise both acyl groups to a similar extent, since yeast cells accumulate twice as much 16:1^{Δ9} as 18:1^{Δ9}. Additionally, the analysis of fatty acids from transgenic yeast cells demonstrated that the introduced KCS from Askari causes the elongation of 18:0 to form 26:0 as the main product. Therefore, the ability of the Askari KCS to elongate C20 and C22 acyl groups seems to be clearly higher with saturated than with monounsaturated acyl-CoA thioesters. Altogether, the data demonstrate that the KCS from Askari is very active in yeast, and that it is also capable to catalyse four to five elongation steps in yeast. In this respect, the KCS from Brassica napus seems to be superior to the KCS from A. thalina which catalyses only two to three elongation steps.

As mentioned before, no VLCFA content could be detected in yeast cells transformed with Drakkar KCS. As already mentioned, the deduced amino acid sequences show only one difference in position 282, the serine in this position in Drakkar being substituted by phenylalanine. This amino acid substitution may yield a catalytically inactive protein, and may therefore cause the LEAR phenotype of the Drakkar variety. This is also verified by data from the analysis of the seed oil from transgenic Drakkar plants, showing that the phenotype with a higher erucic acid content may be reconstituted by introduction of the Askari KCS gene.

Table 3 below shows the fatty acid composition of wild-type, control, and transformed yeast cells. YES2=wild-type control; BnKCSa=yeast cells transformed with Askari BnKCS; BnKCSd=yeast cells transformed with Drakkar BnKCS; BnKCSr=yeast cells transformed with RS306 BnKCS. The values reflect the content of a specific fatty acid as percentage (w/w) of the total fatty acid content.

TABLE 3

| Fatty acid | YES2 | BnKCSa | BnKCSd | BnKCSr |
|---|---|---|---|---|
| 16:0 | 22.83 | 8.51 | 23.78 | 23.08 |
| 16:1^{Δ9} | 45.79 | 31.34 | 44.90 | 44.27 |
| 18:0 | 6.20 | 2.68 | 7.06 | 6.61 |
| 18:1^{Δ9} | 24.00 | 9.17 | 22.97 | 24.55 |
| 18:1^{Δ11} | — | 1.93 | — | — |
| 20:0 | — | 1.87 | — | — |
| 20:1^{Δ11} | — | 0.38 | — | — |
| 22:0 | — | 2.28 | — | — |
| 22:1^{Δ13} | — | 6.87 | — | — |
| 22:1^{Δ15} | — | 11.57 | — | — |
| 24:0 | — | 3.53 | — | — |
| 24:1^{Δ15} | — | 0.86 | — | — |
| 24:1^{Δ17} | — | 3.21 | — | — |
| 26:0 | — | 8.40 | — | — |
| 26:1^{Δ17} | — | 0.30 | — | — |
| 26:1^{Δ19} | — | 1.79 | — | — |

The following FIG. 7 contains data of BnKCSa expression in yeast, with (A) showing several ways of synthesis for various VLCFAs; (B) reflecting the fatty acid content of yeast cells transformed with BnKCSa; and (C) reflecting the increased percentage of various VLCFA species per total fatty acid content.

Lipid extractions and fatty acid analysis were performed according to standard methods, see f.e. Browse et al. (1986) Anal. Biochem. 152: 141-145, the fatty acid methyl ester being further identified by GLC-MS analysis of its nicotinate and di-O-trimethylsilylether derivatives (Dommes et al. (1976) J. Chromatogr. Sci. 14: 360-366; Murata et al. (1978) J. Lipid Res. 19: 172-176).

### Example 6

### Fatty Acid Feeding Experiments with Transgenic Yeast Cells Expressing KCS from B. napus

To analyse the substrate specificities of the KCS from B. napus expressed in yeast cells, feeding experiments with different polyunsaturated fatty acids were conducted. For these experiments, transgenic yeast cells were developed and cultivated as described in Example 5. When the yeast cultures had reached an optical density of 0.5, gene expression was induced by addition of 2% galactose. At this point, various fatty acids were added to the cultures containing 0.1% Tergitol NP-40 to a final concentration of 0.2M, the cultures being further cultivated at 30° C. for 24 hrs (control without addition of fatty acids). Finally, the cells were harvested and used for fatty acid analysis.

It was verified by control experiments, that yeast cells per se are not capable of elongating the employed substrates 18:2^{9,12}, 18:3^{9,12,15}, 18:3^{6,9,12}, 20:3^{8,11,14}, and 20:4^{5,8,11,14}. As shown in Table 4 below, surprisingly, different elongation products were found in yeast cells expressing the KCS from B. napus depending on the employed substrate. These elongation products may be attributed to the activity of the introduced KCS from B. napus.

TABLE 4

| | Substrate | | | | | |
|---|---|---|---|---|---|---|
| | accumulation | Elongation products | | | | Elongation |
| | % total fatty | % total fatty acids | | | | products in |
| Substrate | acids | 20:X | 22:X | 24:X | 26:X | total (%) |
| 18:2^{9,12} | 45,7 | 2,1 | 3,1 | 0,1 | 0,1 | 5,4 |
| 18:3^{9,12,15} | 56,4 | 2,3 | 2,3 | 0,3 | — | 4,9 |
| 18:3^{6,9,12} | 61,3 | 0,7 | 0,1 | 0,1 | 0,4 | 1,3 |
| 20:3^{8,11,14} | 34,1 | — | 3,6 | 0,2 | 0,5 | 4,3 |
| 20:4^{5,8,11,14} | 31,8 | — | 2,2 | 0,1 | 0,6 | 2,9 |

It is obvious from the data summarised in Table 4, that the B. napus KCS expressed in yeast cells utilises the exogenously added fatty acids 18:2^{9,12}, 18:3^{9,12,15}, 18:3^{6,9,12}, 20:3^{8,11,14}, and 20:4^{5,8,11,14}, which were taken up by the yeast cells from the medium, as substrates, and elongates them by 6 to 8 carbons. The products termed 20:X, 22:X, 24:X and 26:X correspond to the expected elongation products. The correct position of the double bonds was verified by GC/MS.

1. A nucleic acid sequence, characterised in that it encodes a protein with the activity of a β-ketoacyl-CoA synthase (KCS) from Brassica napus.

2. The nucleic acid sequence according to claim 1, comprising SEQ ID No. 1 or parts thereof, and encoding a protein with the amino acid sequence in accordance with SEQ No. 1 or parts thereof.

3. A promoter region, characterised in that it naturally controls the expression of a β-ketoacyl-CoA synthase gene.

4. The promoter region according to claim 3, characterised in that it naturally controls the expression of a plant β-ketoacyl-CoA synthase gene.

5. The promoter region according to claim 3 or 4, characterised in that it originates from Brassicaceae, particularly from Brassica napus.

6. The promoter region according to any of claims 3 to 5, comprising SEQ ID No. 2 or parts thereof, which provides for the transcription of an operatively linked coding or non-coding region.

7. A chimeric gene, characterised in that it comprises a promoter region according to any of claims 3 to 6 being operatively linked with a coding region.

8. The nucleic acid molecule, characterised in that it comprises a nucleic acid sequence, a promoter region, or a chimeric gene according to any of the preceding claims.

9. The nucleic acid molecule according to claim 8, characterised in that it comprises a nucleic acid sequence according to claim 1 or 2 being operatively linked with a promoter being active in plants, and especially a seed-specific promoter.

10. The transgenic plants, characterised in that they contain a nucleic acid sequence, a promoter region, a chimeric gene, or a nucleic acid molecule according to any of the preceding claims, as well as parts of these plants and their propagation material, such as protoplasts, plant cells, calli, seeds, tubers, or cuttings as well as the progeny of these plants.

11. The plants according to claim 10 being oil seed plants, particularly rapeseed, turnip rapeseed, sun flower, soy bean, peanut, coco palm, oil palm, cotton, flax.

12. A method of providing seed-specific expression of a coding region in plant seeds, comprising the steps:
a) Generating a nucleic acid sequence, in which a promoter region according to any of the claims 3 to 6 is operatively linked with a coding region,
b) Transferring the nucleic acid sequence from step a) to plant cells, and
c) Regenerating fully transformed plants and, if desired, propagating the plants.

13. The method for shifting the chain length of fatty acids towards longer chain fatty acids in transgenic plants, particularly in oil seed, comprising the steps:
a) Generating a nucleic acid sequence, in which a promoter region being active in plants and particularly in seed tissue is operatively linked with a nucleic acid sequence according to claim 1 or 2,
b) Transfer of the nucleic acid sequence from (a) to plant cells, and
c) Regeneration of fully transformed plants and, if desired, propagation of the plants.

14. The method for increasing the ratio of 22:1 fatty acids to 20:1 fatty acids in transgenic plants, particularly in oil seed, comprising the steps:
a) Generating a nucleic acid sequence in which a promoter region being active in plants and particularly in seed tissue is operatively linked with a nucleic acid sequence according to claim 1 or 2,
b) Transfer of the nucleic acid sequence from (a) to plant cells, and
c) Regeneration of fully transformed plants and, if desired, propagation of the plants.

15. The method for generating longer chain polyunsaturated fatty acids by elongation of shorter chain polyunsaturated fatty acids in microorganisms and plant cells by (i) elongation of naturally occuring polyunsaturated fatty acids, or (ii) elongation of polyunsaturated fatty acids taken up from the environment, comprising the steps:
a) Generating a nucleic acid sequence, in which a promoter region being active in the microorganism or in the plant cell is operatively linked with a nucleic acid sequence encoding a protein with β-ketoacyl-CoA synthase activity,
b) Transfer of the nucleic acid sequence from (a) to microorganisms or plant cells,
c) In the case of plant cells, optionally regeneration of fully transformed plants, and
d) If desired, propagation of the generated transgenic organisms.

16. The method according to claim 15, with the nucleic acid sequence encoding a protein with β-ketoacyl-CoA synthase activity being the nucleic acid sequence according to claim 1 or 2.

17. The method for generating longer chain polyunsaturated fatty acids by elongation of shorter chain polyunsaturated fatty acids in microorganisms and plant cells by elongation of polyunsaturated fatty acids, that are generated in the microorganism or in the plant cell due to the expression of one or more introduced desaturase or/and elongase genes, comprising the steps:
a) Generating a nucleic acid sequence in which a promoter region being active in the microorganism or in the plant cell is operatively linked with a nucleic acid sequence encoding a protein with β-ketoacyl-CoA synthase activity,
b) Transfer of the nucleic acid sequence from (a) to microorganisms or plant cells,
c) In the case of plant cells, optionally regeneration of fully transformed plants, and
d) If desired, propagation of the generated transgenic organisms.

18. The method according to claim 17, with the nucleic acid sequence encoding a protein with β-ketoacyl-CoA synthase activity being a nucleic acid sequence according to claim 1 or 2.

19. The method for changing the β-ketoacyl-CoA synthase activity in transgenic plants by transfer of a nucleic acid sequence according to claim 1 or 2 to plant cells, if desired, with subsequent regeneration of fully transformed plants, and, if desired, propagation of the generated transgenic plants.

20. Use of a promoter region according to any of the claims 3 to 6 for generating transgenic plants, plant cells, plant parts and/or plant products with altered gene expression.

21. Use of a nucleic acid sequence according to claim 1 or 2 for generating transgenic plants, plant cells, plant parts, and/or plant products with an increased 22:1 to 20:1 fatty acid ratio compared to wild-type plants.

22. Use of a nucleic acid sequence according to claim 1 or 2 for generating transgenic plants, plant cells, plant parts, and/or plant products with a fatty acid pattern that is shifted towards longer chain fatty acids compared to wild-type plants.

23. Use of a nucleic acid sequence encoding a protein with β-ketoacyl-CoA synthase activity for generating transgenic microorganisms or plant cells with a pattern of polyunsaturated fatty acid that is shifted towards longer chain fatty acids compared to the original forms.

24. The use according to claim 23, the nucleic acid sequence being a nucleic acid sequence according to claim 1 or 2.

25. A promoter region, characterized in that it naturally controls the expression of a plant β-ketoacyl-CoA synthase gene and has a nucleotide sequence, which
is comprised by the sequence shown in SEQ ID No. 2 and comprises both the promoter elements TATA box and CAAT box, or
hybridizes with the promoter region shown in SEQ ID No. 2 under stringent hybridization conditions, or
shows at least 70-80% sequence identity with the promoter region shown in SEQ ID No. 2.

26. The promoter region according to claim 25, characterized in that its nucleotide sequence is comprised by the sequence shown in SEQ ID No. 2, comprises both the promoter elements TATA box and CAAT box, and hybridizes with the promoter region shown in SEQ ID No. 2 under stringent hybridization conditions.

27. The promoter region according to claim 25, characterized in that its nucleotide sequence is comprised by the sequence shown in SEQ ID No. 2, comprises both the promoter elements TATA box and CAAT box, and shows at least 70-80% sequence identity with the promoter region shown in SEQ ID No. 2.

28. The promoter region according to claim 25, characterized in that its nucleotide sequence hybridizes with the promoter region shown in SEQ ID No. 2 under stringent hybridization conditions, and shows at least 70-80% sequence identity with the promoter region shown in SEQ ID No. 2.

29. The promoter region according to claim 25, characterized in that its nucleotide sequence is comprised by the sequence shown in SEQ ID No. 2, comprises both the promoter elements TATA box and CAAT box, hybridizes with the promoter region shown in SEQ ID No. 2 under stringent hybridization conditions, and shows at least 70-80% sequence identity with the promoter region shown in SEQ ID No. 2.

30. The promoter region according to claim 25, characterized in that its nucleotide sequence comprises an RY-repeat (CATGCATG) between the CAAT box and the TATA box, and/or an E-box (CACATG) next to the TATA box.

31. The promoter region according to claim 25, characterized in that it originates from Brassicaceae, particularly from Brassica napus.

32. A chimeric gene, characterized in that it comprises a promoter region according to any of the preceding claims being operatively linked with a coding region.

33. A nucleic acid molecule, characterized in that it comprises a promoter region or a chimeric gene according to claim 25.

34. A transgenic plant, characterized in that it contains a promoter region, a chimeric gene, or a nucleic acid molecule according to any of the preceding claims, as well as parts of said plant and its propagation material, such as protoplasts, plant cells, calli, seeds, tubers, and cuttings as well as its progeny.

35. The plant according to claim 34 being an oil seed plant, particularly rapeseed, turnip rapeseed, sun flower, soy bean, peanut, coco palm, oil palm, cotton or flax.

36. A method of providing seed-specific expression of a coding region in plant seeds, comprising the steps:
a) Generating a nucleic acid sequence, wherein a promoter region according to any of the claims 25 to 31 is operatively linked with a coding region,
b) Transferring the nucleic acid sequence from step a) to plant cells, and
c) Regenerating fully transformed plants and, if desired, propagating the plants.

37. Use of a promoter region according to any of the claims 25 to 31 for generating transgenic plants, plant cells, plant parts and/or plant products with altered gene expression.
